(19) United States
(12) Patent Application Publication
(10) Pub. No.: US 2026/0174854 A1
(43) Pub. Date: Jun. 25, 2026

(54) Systems And Methods For Imaging Diverse Pathogenic Bacteria In Vivo With [18F]Fluoromannitol Positron Emission Tomography

(71) Applicants: Kiel NEUMANN, Olive Branch, MS (US); Spenser SIMPSON, Olive Branch, MS (US); UNIVERSITY OF VIRGINIA PATENT FOUNDATION, Charlotteville, VA (US); ST. JUDE CHILDREN'S RESEARCH HOSPITAL, INC., Memphis, TN (US)

(72) Inventors: Kiel Neumann, Olive Branch, MS (US); Spenser Simpson, Olive Branch, MS (US)

(21) Appl. No.: 18/880,130

(22) PCT Filed: Jul. 3, 2023
(86) PCT No.: PCT/US2023/026841

§ 371 Date: Dec. 30, 2024

Related U.S. Application Data
(60) Provisional application No. 63/357,716, filed on Jul. 1, 2022.
(60) Provisional application No. 63/524,464, filed on Jun. 30, 2023.

(51) Int. Cl. A61K 45/06; A61K 9/00; A61K 51/08

(52) U.S. Cl. CPC A61K 45/06; A61K 9/0019; A61K 51/088

(57) ABSTRACT

Compositions for identifying a pathogenic bacterial infection include a mannitol compound with one or more substituents including radioisotopes. These radiopharmaceuticals, such as a positron-emitting mannitol analogue, [¹⁸F]fluoromannitol ([¹⁸F]FMtl), are rapidly taken up by gram-positive and gram-negative bacteria such as E. coli, S. aureus, A. baumannii, S. epidermis, P. mirabilis, S. enterica, K. pneumonia, E. faecium, E. cloacae, and M. marinum, but not non-active infection sites such as sterile inflammatory sites, cancer sites, etc. Administration of these radiopharmaceuticals to and subsequent imaging of patients, e.g., via positron emission tomography (PET), enables detection of deep-seated and difficult to manage bacterial infections, such as osteomyelitis and prosthetic joint infection, or in patients with sickle cell disease. [¹⁸F]FMtl injection detects and differentiates infection rapidly. The radiolabeled mannitol compounds can be produced via nucleophilic substitution reactions that are deployable on commercially available synthesizers, facilitating straightforward and wide accessibility, and counteracting unnecessary antibiotic use.

### CROSS REFERENCE TO RELATED APPLICATION(S)

This application claims the benefit of U.S. Provisional Application Nos. 63/357,716, filed Jul. 1, 2022, and 63/524,464, filed Jun. 30, 2023, which are incorporated by reference as if disclosed herein in their entireties.

### STATEMENT REGARDING FEDERALLY SPONSORED RESEARCH OR DEVELOPMENT

This invention was made with government support under N3239820P0034 awarded by the Department of Defense, and EB028338 awarded by the National Institutes of Health. The government has certain rights in the invention.

### BACKGROUND

Infection is responsible for the highest morbidity and the third most deaths among all human diseases worldwide. The majority of healthcare-associated infections in the United States arise from several common pathogens, including S. aureus, A. baumannii, P. aeruginosa, and those of the Enterobacteriaceae family (E. coli, Salmonella spp., et al.). The rising trend of antimicrobial resistance, compounded by a growing population of immunocompromised individuals (HIV/AIDS, chemotherapy, organ transplant, diabetes) creates an enormous economic strain on the United States healthcare system, with estimates ranging from $28-$45B annually. Current estimates project that drug-resistant infections will become the leading cause of global death, surpassing cancer-associated mortality by 2050. Accordingly, there is an urgent need to improve the clinical management, including diagnosis and treatment, of bacterial infection. The CDC recently listed carbapenem-resistant Acinetobacter and Enterobacteriaceae, ESBL-producing Enterobacteriaceae, multidrug-resistant P. aeruginosa, methicillin-resistant S. aureus, and others as Urgent or Serious Threats to human health. Carbapenem-resistant Acinetobacter baumannii (CrAB) alone was responsible for 8,500 hospitalizations, 700 deaths, and $281M in US healthcare costs in 2017. CrAB infections are particularly problematic for patients with co-morbidities and the immunocompromised; however, A. baumannii-associated infections are also well-described complications of severe combat-related injuries suffered by military service members. Accordingly, there is an urgent need to improve the diagnosis and treatment of bacterial infection.

Traditional approaches for diagnosing infection include sample biopsy from tissue or blood and subsequent culture of pathogens in media for an organism's identification. Bacterial culture from tissue biopsy remains the “gold standard” for confirming the presence, identity, and drug sensitivity of a microorganism; however, deep-seated infections that are difficult to access or identify often rely upon non-invasive imaging techniques based on changes in anatomy or tissue morphology. The most common anatomical imaging modalities used, such as computed tomography (CT) and magnetic resonance imaging (MRI), are frequently nonspecific for delineating active infection from sterile inflammatory disease. Nuclear medicine utilizes labeled leukocytes ([^{99m}Tc]- or [¹¹¹In]-oxine) and [⁶⁷Ga]-citrate scintigraphy, which relies upon indirect measurements of leukocyte recruitment to an area of interest. Positron emission tomography (PET) imaging with 2-[¹⁸F]fluoro-2-deoxy-glucose ([¹⁸F]FDG) is increasingly used; however, none of these imaging techniques are able to distinguish active infection from cancer or inflammation. Consequently, current clinically-available imaging techniques are not adequately specific to diagnose deep-seated infection.

To address this challenge, many recently developed radiopharmaceuticals seek to exploit various bacteria-specific signatures such as metabolism, cofactor biosynthesis, and labeled antibiotics. Despite these scientific advances, a dire need persists for imaging agents that meet the challenges of clinical infectious diseases practice; such an agent should possess broad bacterial strain sensitivity, advantageous PK (rapid target engagement, clearance of nonspecific signals to promote contrast), and wide deployability/availability for clinical use.

### SUMMARY

Aspects of the present disclosure are directed to a composition for identifying a pathogenic bacterial infection. In some embodiments, the composition includes a mannitol compound including one or more substituents, wherein at least one of the substituents includes a radioisotope. In some embodiments, the mannitol compound includes a chemical structure according to Formula II:

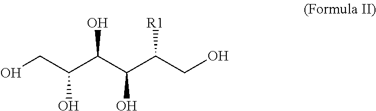

In some embodiments, R1 includes at least one radioisotope. In some embodiments, the at least one radioisotope includes a halogen isotope. In some embodiments, the at least one radioisotope includes [¹⁸F]. In some embodiments, the mannitol compound includes a chemical structure according to Formula III:

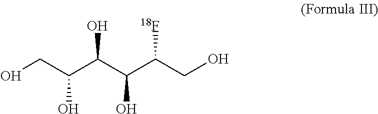 [SMILES: OC[C@@H](O)[C@@H](O)[C@H](O)[C@H]([18F])CO]

In some embodiments, the composition includes one or more additional active ingredients, or one or more pharmaceutically acceptable adjuvants, diluents, excipients, carriers, or combinations thereof.

Aspects of the present disclosure are directed to a method of treating a pathogenic bacterial infection in a patient. In some embodiments, the method includes preparing a composition including a concentration of a mannitol compound including one or more substituents, wherein at least one of the substituents includes a radioisotope; administering an amount of the composition to the patient; imaging one or more tissues of the patient; and identifying elevated concentrations of radioisotope in an imaged tissue. In some embodiments, the method includes administering an effective amount of an antibiotic after elevated concentrations of radioisotope have been identified. As discussed above, in some embodiments, the radioisotope includes a halogen isotope. In some embodiments, the radioisotope includes [¹⁸F]. In some embodiments, the mannitol compound includes a chemical structure according to Formula III:

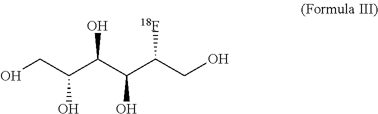 [SMILES: OC[C@@H](O)[C@@H](O)[C@H](O)[C@H]([18F])CO]

In some embodiments, imaging one or more tissues of the patient includes Positron Emission Tomography (PET). In some embodiments, the pathogenic bacterial infection includes E. coli, S. aureus, A. baumannii, S. epidermis, P. mirabilis, S. enterica, K. pneumonia, E. faecium, E. cloacae, M. marinum, or combinations thereof. In some embodiments, the patient is positive for sickle cell disease.

Aspects of the present disclosure are directed to a method of making a composition for identifying a pathogenic bacterial infection. In some embodiments, the method includes providing a reaction medium including a concentration of a mannose compound including one or more substituents, wherein at least one of the substituents includes a radioisotope; and converting the mannose compound to a mannitol compound via hydride-mediated reduction of the mannose compound. In some embodiments, providing a reaction medium including a concentration of a mannose compound including one or more substituents includes providing a precursor reaction medium including a concentration of a precursor compound including 4,6-O-Benzylidene-3-O-ethoxymethyl-2-O-trifluoromethanesulfonyl-1-O-methyl-β-D-glucopyranoside; and forming the mannose compound by performing a substitution reaction at at least one carbon in the precursor compound to attach the radioisotope as a substituent of the at least one carbon. As discussed above, in some embodiments, the radioisotope includes a halogen isotope. In some embodiments, the radioisotope includes [¹⁸F]. In some embodiments, the mannose compound includes 2-[¹⁸F]fluoro-2-deoxy-mannose ([¹⁸F]FDM). In some embodiments, the mannitol compound includes the chemical structure according to Formula III.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1A is a graph showing accumulation of [¹⁸F]fluoromannitol ([¹⁸F]FMtl) in S. aureus cultures;

FIG. 1B is a graph showing uptake of [¹⁸F]FMtl in pathogens of clinical interest, determined at 60 minutes of incubation;

FIG. 1C is a graph showing a competitive uptake assay of [¹⁸F]FMtl in the presence of unlabeled D-mannitol in S. aureus;

FIG. 1D is a graph showing in vitro gamma counts of S. aureus and E. coli cultures incubated with [¹⁸F]FMtl, 2-[¹⁸F]fluoro-2-deoxy-glucose ([¹⁸F]FDG), and 2-[¹⁸F]fluoro-2-deoxy-sorbitol ([¹⁸F]FDS);

FIG. 2A shows imaging data indicating a significantly elevated [¹⁸F]FMtl signal in infected triceps brachii but not present in inflamed triceps brachii in both S. aureus (left) and E. coli (middle), while [¹⁸F]FDG is unable to differentiate infection from sterile inflammation (right);

FIG. 2B is a graph showing computed standard uptake values (SUV) of [¹⁸F]FMtl and [¹⁸F]FDG in a murine myositis model of infection;

FIG. 2C is a graph showing [¹⁸F]FMtl imaging (SUV or Patlak slope (K;) (rate of tracer uptake)) correlated with bacteria colony forming units (CFU) ex vivo to demonstrate imaging agent sensitivity;

FIG. 2D is an image showing the improved imaging sensitivity of [¹⁸F]FMtl using parametric imaging (left) compared to the clinical standard metric of SUV (right);

FIGS. 3A-3B portray images and data showing the advantageous positron emission tomography imaging (PET)/CT static imaging of a murine mixed myositis model of infection utilizing compositions according to some embodiments of the present disclosure;

FIG. 4A is a chart of a method of making a composition for identifying a pathogenic bacterial infection according to some embodiments of the present disclosure;

FIG. 4B is a chart of a method of making a composition for identifying a pathogenic bacterial infection according to some embodiments of the present disclosure;

FIG. 5 is a flowchart showing a reaction scheme for making a mannitol compound according to some embodiments of the present disclosure, and characteristic high-performance liquid chromatography (HPLC) data thereof,

FIG. 6 is a chart of a method of treating a pathogenic bacterial infection in a patient according to some embodiments of the present disclosure;

FIG. 7A portrays an image and graph showing antimicrobial efficacy as quantified with serial PET imaging (left) and correlated with CFU (right); and

FIG. 7B is a graph showing uptake of [¹⁸F]FMtl in clinical isolates of S. aureus, A. Baumannii, and P. aeruginosa.

### DETAILED DESCRIPTION

Some embodiments of the present disclosure is directed to a composition for identifying a pathogenic bacterial infection in an individual, e.g., a patient. In some embodiments, the composition includes one or more radiopharmaceuticals. In some embodiments, the radiopharmaceutical is configured to be taken up by one or more bacteria at an infection site in or on the individual, e.g., an infection of one or more tissues of the individual. In some embodiments, the one or more bacteria includes E. coli, S. aureus, A. baumannii, S. epidermis, P. mirabilis, S. enterica, K. pneumonia, E. faecium, E. cloacae, M. marinum, or combinations thereof, or any other bacteria responsible for or contributing to an infection in the individual, or combinations thereof.

In some embodiments, the radiopharmaceutical is a radiolabeled compound. In some embodiments, the compound is a polyalcohol. In some embodiments, the compound includes a chemical structure according to Formula I:

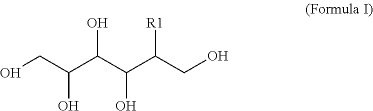

In some embodiments, R1 includes at least one radioisotope. In some embodiments, R1 includes a plurality of radioisotopes. In some embodiments, the compound is a derivative of Formula I, e.g., includes one or more additional substituents, is bound to one or more additional chemical structures, etc.

In some embodiments, the compound includes a mannitol compound or a derivative thereof. In some embodiments, the radiopharmaceutical is a mannitol compound including one or more substituents. In some embodiments, the radiopharmaceutical is a mannitol compound including a plurality of substituents. In some embodiments, at least one of the substituents includes a radioisotope. In some embodiments, at least one of the substituents includes a plurality of radioisotopes. In some embodiments, the radioisotope includes a halogen isotope. In some embodiments, the radioisotope includes a fluorine isotope. In some embodiments, the radioisotope includes [¹⁸F].

In some embodiments, the mannitol compound includes a chemical structure according to Formula II:

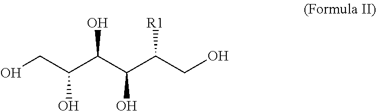

In some embodiments, R1 includes at least one radioisotope. In some embodiments, R1 includes a plurality of radioisotopes. In some embodiments, the mannitol compound is a derivative of Formula II, e.g., includes one or more additional substituents, is bound to one or more additional chemical structures, etc.

In some embodiments, the mannitol compound includes a chemical structure according to Formula III:

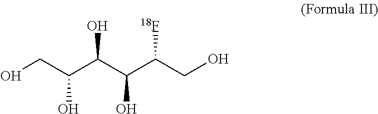 [SMILES: OC[C@@H](O)[C@@H](O)[C@H](O)[C@H]([18F])CO]

In some embodiments, the mannitol compound is a derivative of Formula III, e.g., includes one or more additional substituents, is bound to one or more additional chemical structures, etc. In some embodiments, the composition includes one or more compounds according to Formula III, derivatives thereof, or combinations thereof. As used herein, the chemical structure according to Formula III and the term [¹⁸F]fluoromannitol ([¹⁸F]FMtl) are used interchangeably.

In some embodiments, the composition includes one or more additional compounds for use with a particular treatment of a pathogenic bacterial infection in an individual. In some embodiments, the composition includes one or more additional active ingredients. In some embodiments, the composition includes one or more pharmaceutically acceptable adjuvants, diluents, excipients, carriers, or combinations thereof.

In some embodiments, the radiopharmaceutical is preferentially taken up at a site of active infection, e.g., taken up by actively infectious bacteria, as compared to non-active infection sites, e.g., sterile inflammatory sites, cancer sites, etc. In some embodiments, the radiopharmaceutical preferentially binds to the surface of bacteria at a site of active infection as compared to non-active infection sites, e.g., sterile inflammatory sites, cancer sites, etc. In some embodiments, the radiopharmaceutical facilitates preferential uptake and/or binding of radioisotopes by bacteria at a site of active infection as compared to non-active infection sites, e.g., sterile inflammatory sites, cancer sites, etc. In some embodiments, the radiopharmaceutical is retained by the actively infectious bacteria at the infection site. In some embodiments, retention of the radiopharmaceutical in or on the individual is temporary, e.g., the radiopharmaceutical is able to be evacuated from the individual after a predetermined period of time. In some embodiments, the predetermined period of time is sufficient to perform an imaging process on one or more tissues of the individual having or suspected of having the active infection, as will be discussed in greater detail below. In some embodiments, the radiopharmaceutical is taken up by the bacteria at the infection site via the phosphoenolpyruvate-dependent sugar phosphotransferase system (sugar PTS) of the bacteria.

Mannitol compounds consistent with embodiments of the present disclosure specifically accumulate in both gram-positive and gram-negative bacteria but not in mammalian cells, both in vitro and in vivo. The sugar PTS catalyzes phosphorylation of incoming sugar substrates with concomitant translocation across the cell membrane and is widely found in bacteria, including E. coli, A. baumannii, P. aeruginosa, and many gram-positive organisms.

Without wishing to be bound by theory, the diagnostic efficacy of a radiotracer is ultimately limited by its ability to promote sufficient contrast, which is enhanced by minimizing nonspecific accumulation in normal tissue. Many different bacteria-specific imaging agents have been studied to date; these agents exploit divergent prokaryotic mechanisms and display varying degrees of background contamination in positron emission tomography imaging (PET)-derived images. For example, folate biosynthesis-targeted imaging agents show increased signal in the gallbladder and spine, limiting this tracer's utility for difficult to manage, clinically-relevant infections like discitis osteomyelitis. While D-amino acid-derived radiotracers demonstrate impressive “broad spectrum” bacterial uptake, these agents accumulate significantly in the liver and GI tract. In addition, these imaging agents utilize the PET isotope, carbon-11; due to the short half-life (t_{1/2}=20 minutes) of this isotope, the clinical utility is limited to sites with direct cyclotron access.

In an exemplary embodiment, [¹⁸F]FMtl was able to differentiate sterile inflammation and bacterial infection resulting from S. aureus and E. coli in vivo in a murine myositis model using PET imaging. Exemplary embodiments of the present disclosure were extended to a laceration wound model infected with A. baumannii, an important pathogen in the nosocomial and battlefield setting. [¹⁸F]FMtl PET rapidly and specifically detected infections caused by A. baumannii and several other important ESKAPE pathogens. [¹⁸F]FMtl PET was also able to monitor therapeutic efficacy of vancomycin against S. aureus in vivo. The ease of production of [¹⁸F]FMtl facilitates wide dissemination from any radiopharmacy. Furthermore, the broad specificity for bacterial infection in vivo demonstrates that [¹⁸F]FMtl is a suitable imaging agent for human use.

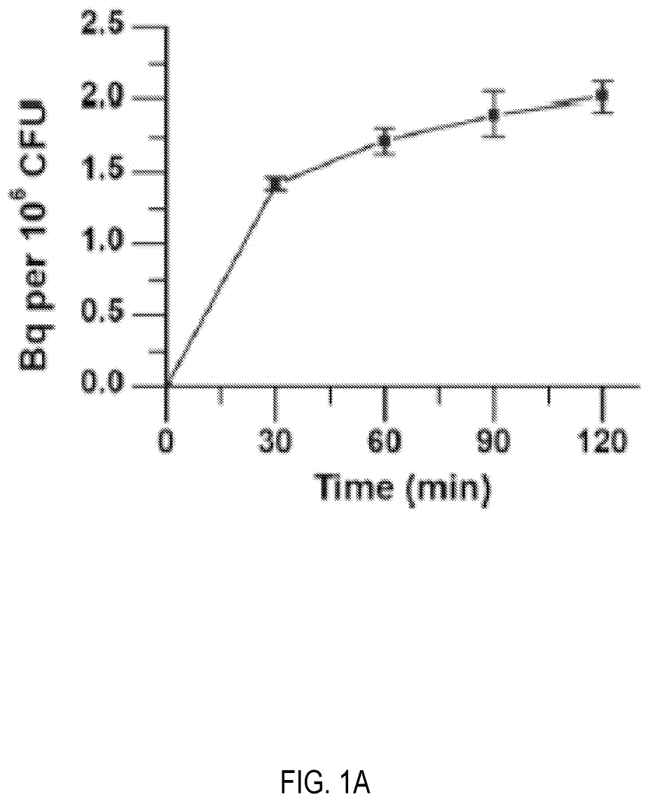

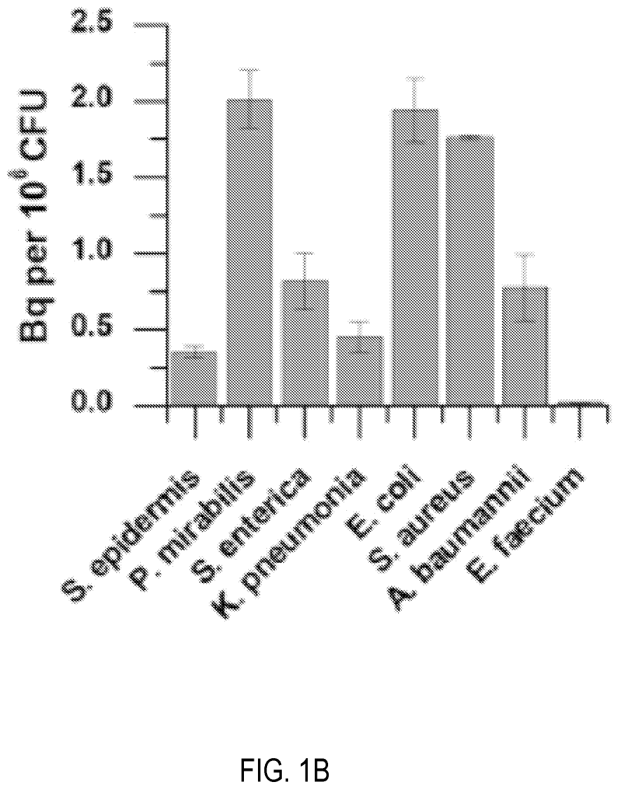

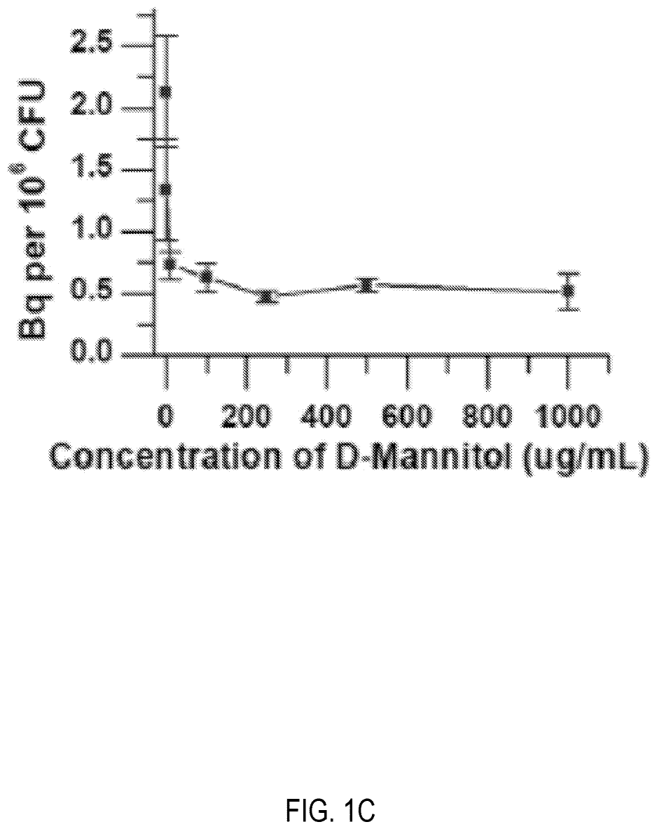

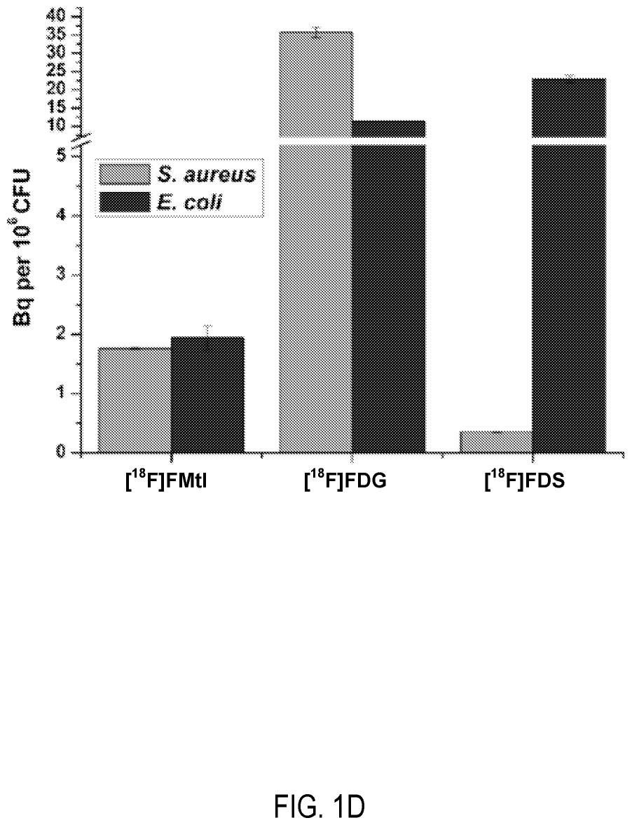

Referring now to FIGS. 1A-1D, in an exemplary embodiment, both S. aureus and E. coli readily incorporated [¹⁸F]FMtl over time (see FIG. 1A), but not heat-killed bacteria, demonstrating metabolic specificity of bacteria for [¹⁸F]FMtl. Accumulation of [¹⁸F]FMtl was also evaluated in a broad panel of bacterial strains. All strains tested, except for P. aeruginosa and E. faecium, showed rapid and significant accumulation (see FIG. 1B). Co-incubation of [¹⁸F]FMtl with D-mannitol in S. aureus cultures demonstrated target specificity and that accumulation of [¹⁸F]FMtl is not concentration-dependent (see FIG. 1C); concentrations of ≥10 μg/mL of D-mannitol blocked [¹⁸F]FMtl accumulation in bacteria. The accumulation of [¹⁸F]FMtl in S. aureus (gram-positive) and E. coli (gram-negative) cultures was also compared against 2-[¹⁸F]fluoro-2-deoxy-glucose ([¹⁸F]FDG), the current workhorse of nuclear medicine, and 2-[¹⁸F]fluoro-2-deoxy-sorbitol ([¹⁸F]FDS), which has demonstrated high specificity for Enterobacteriaceae organisms. As shown, [¹⁸F]FMtl was accumulated in both E. coli and S. aureus, which was significantly higher in S. aureus compared to [¹⁸F]FDS (P<0.001) (see FIG. 1D). [¹⁸F]FMtl accumulation in S. aureus and E. coli was not significantly different (P=0.64); whereas, [¹⁸F]FDS accumulation in S. aureus was not significantly different to negative control. Taken together, these data show [¹⁸F]FMtl is rapidly accumulated in a wide panel of bacteria; thus, useful to serve as a broad-spectrum imaging agent of infection in vivo.

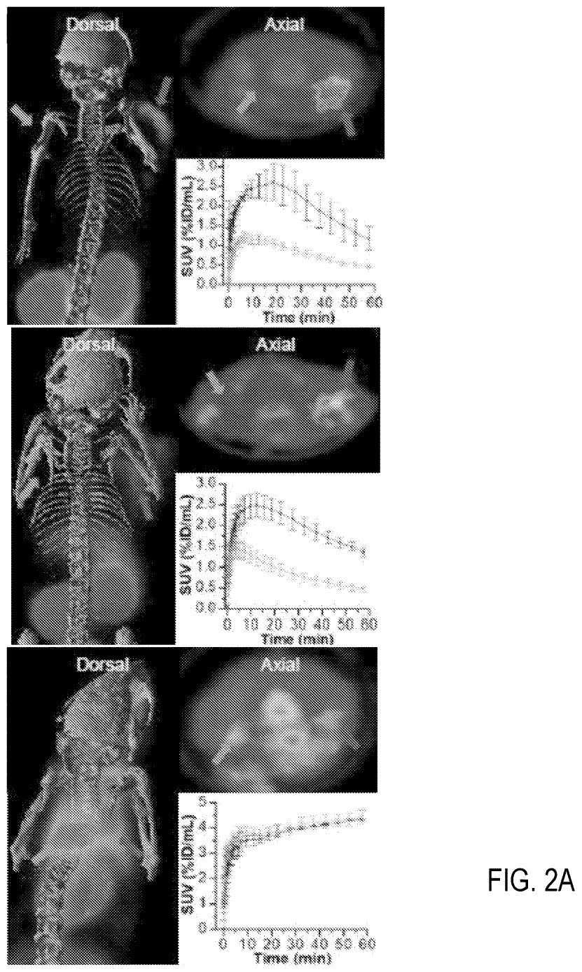

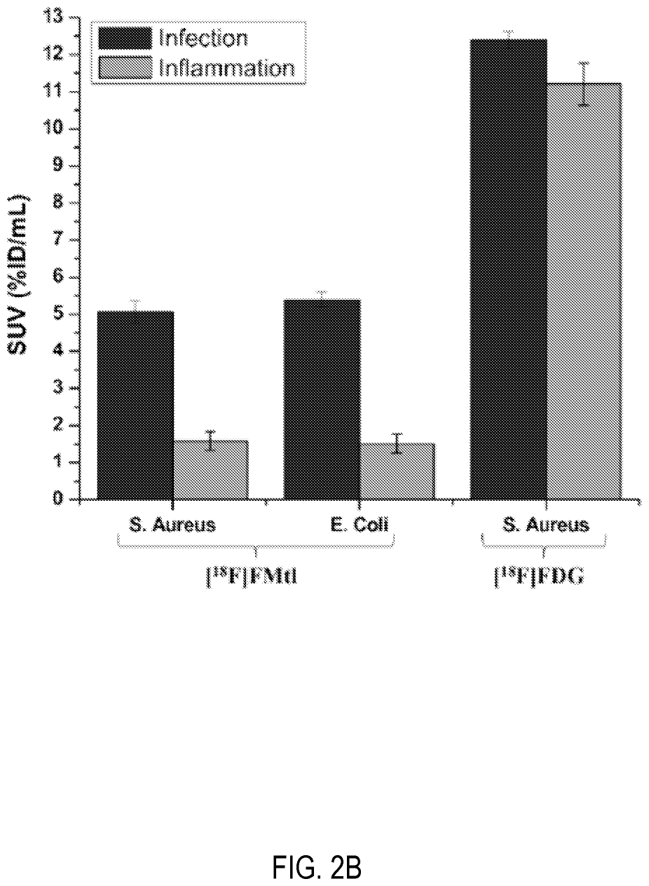

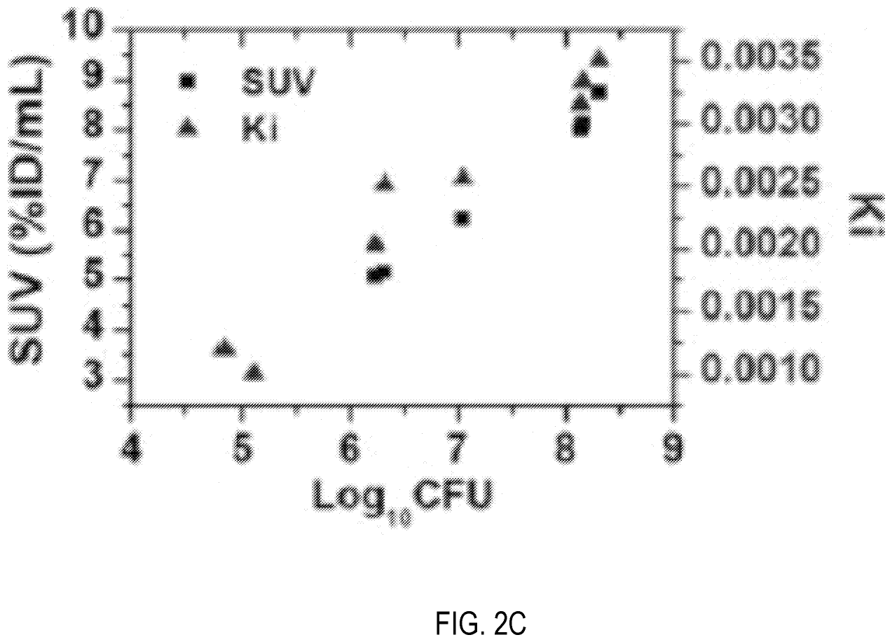

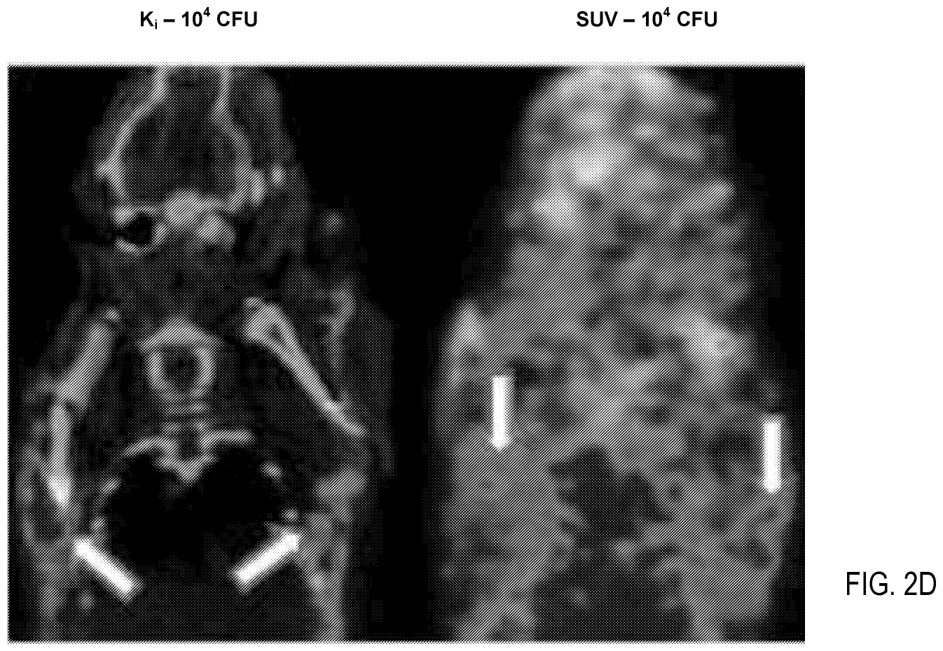

Referring now to FIGS. 2A-2D, a murine myositis model of musculoskeletal infection was used to determine whether [¹⁸F]FMtl could differentiate sterile inflammation from infection in vivo by inoculating the right triceps brachii with a live strain of bacteria and the left triceps brachii with 10× heat-killed bacteria to generate an inflammatory response. [¹⁸F]FMtl was specifically accumulated in the site of infection in both gram-positive and gram-negative strains (see FIG. 2A). [¹⁸F]FDG was unable to distinguish infection from inflammation. Dynamic imaging revealed rapid accumulation and significant differences in PET signal in as little as 5 minutes post-[¹⁸F]FMtl injection.

To quantify PET signal, volumes of interest (VOIs) were generated in the upper limbs of mice using CT for anatomical localization. [¹⁸F]FMtl displayed 3.5-fold increased standardized uptake value (SUV) (summed frames 45-60 minutes post-injection) compared to the contralateral site of inflammation (see FIG. 2B). [¹⁸F]FDG was unable to demonstrate significant differences in SUV between sites of infection and inflammation. After scans, both triceps brachii were excised to confirm PET data using gamma counting, which confirmed the increased PET signal in the infected tissue compared to inflamed. Biodistribution studies were performed in successive cohorts of mice over 3 hours to determine dosimetry of [¹⁸F]FMtl. Kidneys and bladder demonstrated highest nonspecific accumulation of [¹⁸F]FMtl, consistent with PET imaging data. Static PET SUV were correlated with bacterial colony-forming units (CFU) from excised tissue to determine sensitivity of [¹⁸F]FMtl (see FIG. 2C), indicating [¹⁸F]FMtl can reliably detect as little as 5 log₁₀ (CFU/mL) of bacteria in vivo by SUV. It was also investigated whether parametric mapping could increase bacterial sensitivity of [¹⁸F]FMtl in vivo, as Patlak slope (K,) is a quantitative measure of rate of uptake in tissue, rather than SUV, which is semi-quantitative and cannot delineate signal from blood pool contamination and tissue. Parametric imaging improved the bacterial sensitivity of [¹⁸F]FMtl by roughly 20-fold (log₁₀ CFU=1.3; 1.7×10⁶ improved to 7.0×10⁵ CFU) (see FIG. 2D).

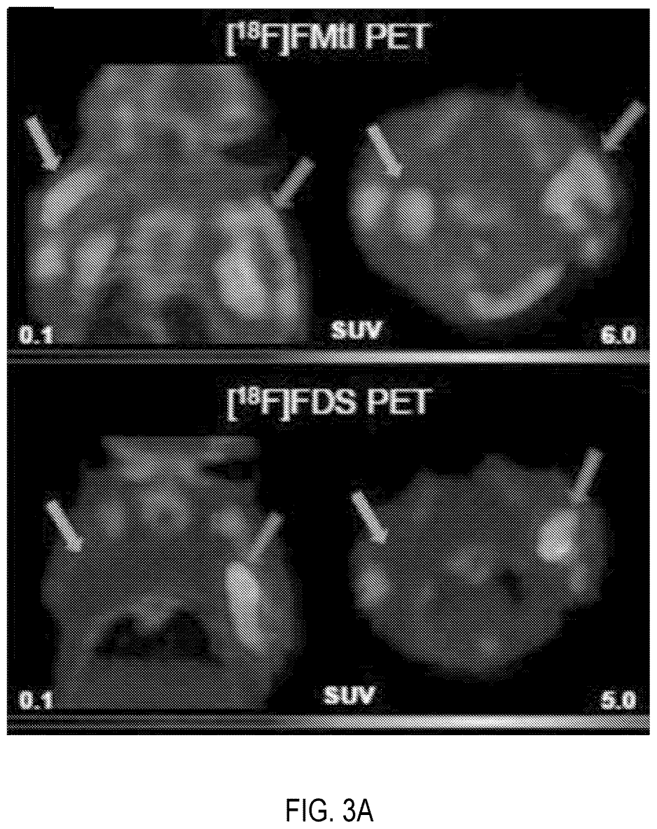

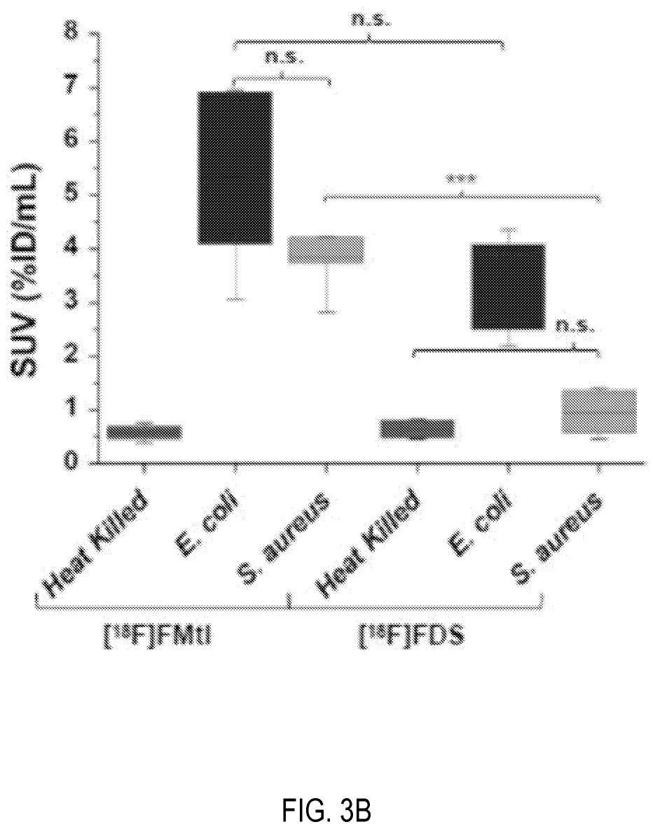

Referring now to FIGS. 3A-3B, the sensitivity of [¹⁸F]FMtl compared to [¹⁸F]FDS was also investigated in a mixed infection (polymicrobial) model. [¹⁸F]FDS has shown remarkable specificity for Enterobacteriaceae in vivo, but has limited to no sensitivity toward gram-positive and other gram-negative organisms. Mice were inoculated with live E. coli (8.4×10⁶ CFU) and S. aureus (8.8×10⁶ CFU) in the right and left triceps brachii, respectively. No significant differences in SUV (P=0.19) were observed between E. coli (orange arrow) and S. aureus (blue arrow) infection in the same animal (see FIGS. 3A-3B) with [¹⁸F]FMtl. Importantly, [¹⁸F]FMtl accumulation was significantly higher compared to [¹⁸F]FDS in S. aureus infection (P<0.001). [¹⁸F]FDS demonstrated high specificity for E. coli compared to S. aureus (P=0.007); however, no significant differences in E. coli SUV were evident between [¹⁸F]FMtl and [¹⁸F]FDS (P=0.11). Postmortem gamma counting of tissues confirmed [¹⁸F]FMtl uptake in both S. aureus and E. coli, where [¹⁸F]FDS only accumulated in E. coli-infected muscle. These data show [¹⁸F]FMtl is accumulated in both gram-positive and gram-negative organisms and exhibits the sensitivity to serve as a broad-spectrum imaging tool for infection in vivo.

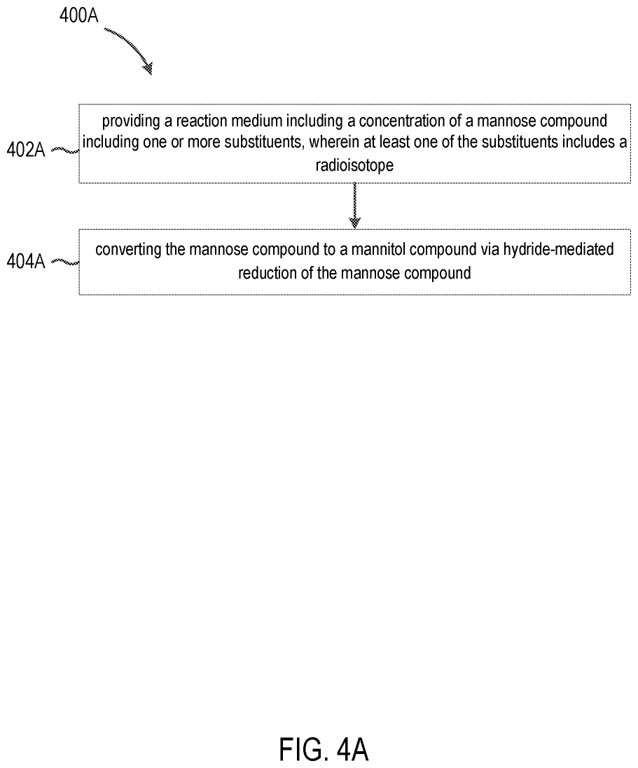

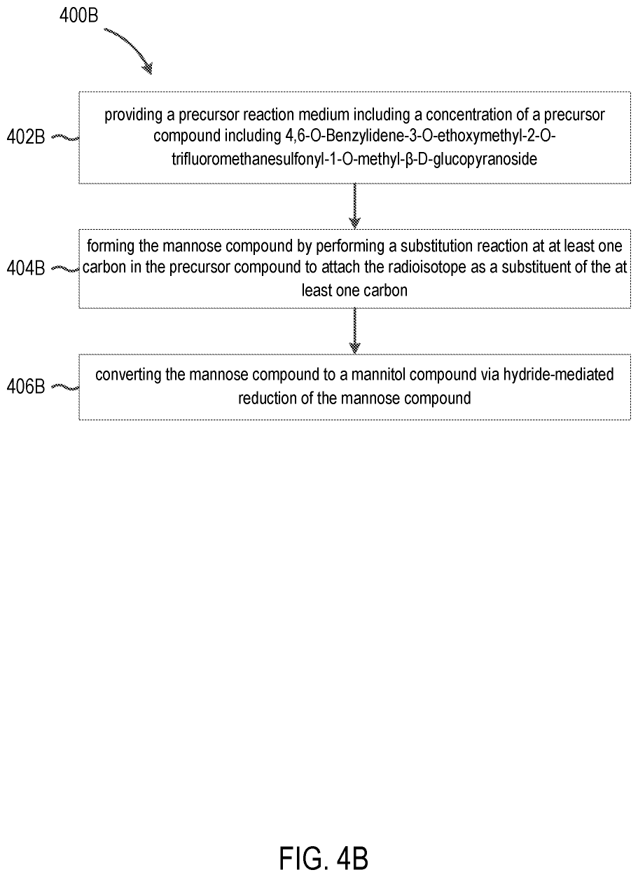

Referring now to FIGS. 4A-4B, in some embodiments, the present disclosure is directed to methods (400A and 400B) of making a composition for identifying a pathogenic bacterial infection. Referring specifically to FIG. 4A, at 402A, a reaction medium was provided including a concentration of a mannose compound. In some embodiments, the mannose compound includes one or more substituents. In some embodiments, the mannose compound includes a plurality of substituents. In some embodiments, at least one of the substituents includes a radioisotope. In some embodiments, at least one of the substituents includes a plurality of radioisotopes. As discussed above, in some embodiments, the radioisotope includes a halogen isotope. In some embodiments, the radioisotope includes a fluorine isotope. In some embodiments, the radioisotope includes [¹⁸F]. In some embodiments, the mannose compound includes 2-[¹⁸F]fluoro-2-deoxy-mannose ([¹⁸F]FDM).

At 404A, at least a portion of the mannose compound in the reaction medium is converted to a mannitol compound. In some embodiments, the mannitol compound is consistent with those discussed above. In some embodiments, the mannitol compound includes the chemical structure according to Formula III above. In some embodiments, the mannose compound is converted 404A via hydride-mediated reduction of the mannose compound. In some embodiments, the mannose compound is converted 404A via sodium borohydride-mediated reduction of the mannose compound, e.g., at the anomeric carbon of [¹⁸F]FDM after an intermediate sep-pak purification. In some embodiments, the mannose compound is first converted to an intermediate compound, and subsequently converted from the intermediate compound to the mannitol compound via one or more reaction steps.

Referring now to FIG. 4B, in some embodiments, providing a reaction medium including a concentration of a mannose compound, e.g., step 402A above, includes the step of providing 402B a precursor reaction medium including a concentration of a precursor compound for conversion into a mannose compound. In some embodiments, the precursor compound includes 4,6-O-Benzylidene-3-O-ethoxymethyl-2-O-trifluoromethanesulfonyl-1-O-methyl-β-D-glucopyranoside. In some embodiments, at 404B, the mannose compound is formed by performing a substitution reaction at at least one carbon in the precursor compound to attach the radioisotope as a substituent of the at least one carbon. In some embodiments, the mannose compound is formed 404B by first converting the precursor compound into one or more intermediate compounds, which are subsequently converted from the intermediate compound to the mannose compound. Then at 406B, as above with respect to converting step 404A, the mannose compound is converted to a mannitol compound, e.g., Formula III above.

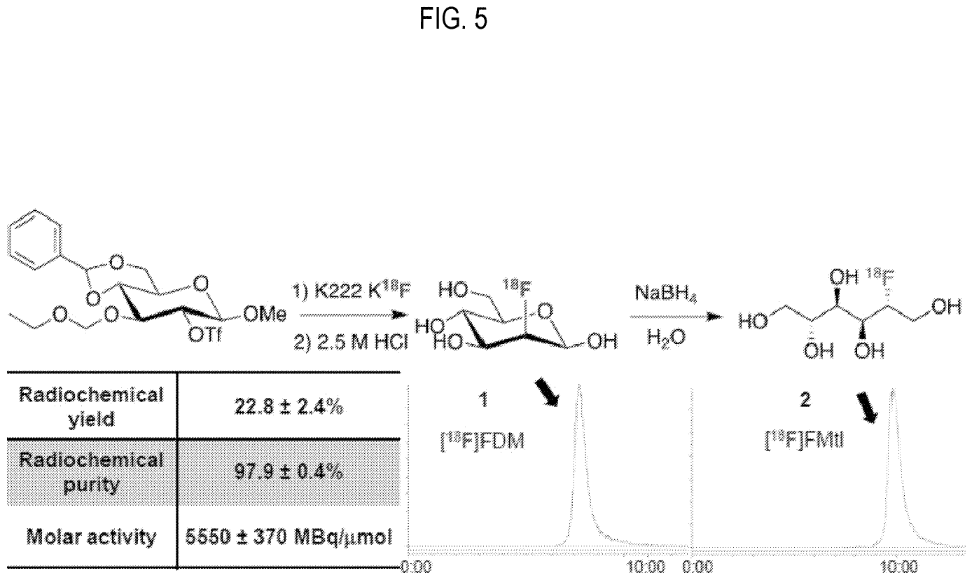

Referring now to FIG. 5, the use of fluorine-18 ensures the isotope is regularly available from cyclotron production, and the half-life (t_{1/2}=109.5 minutes) facilitates widespread distribution. In some embodiments, the radiosynthesis of [¹⁸F]FMtl is performed via a 3-step reaction process. In some embodiments, the first two steps model the radiosynthesis of [¹⁸F]FDG, followed by sodium borohydride reduction. In some embodiments, purifications are cartridge-based, facilitating automation on radiosynthesizers and promoting robust access to [¹⁸F]FMtl. Exemplary embodiments of the radiosynthesis according to the present disclosure produce [¹⁸F]FMtl in high radiochemical yields and purity, which are easily determined by radioHPLC. Fully protected ¹⁹F-isotopic precursor of [¹⁸F]FDM were synthesized to identity the [¹⁸F]-labeled intermediate and calculate molar activity by HPLC. The production of [¹⁸F]FMtl generated 7.31 f 0.25 μg/mL of [¹⁹F]FMtl, which meets the FDA “microdose” definition and is suitable for clinical studies. [¹⁸F]FDM was converted to [¹⁸F]FMtl by sodium borohydride-mediated reduction and isolated in >99% radiochemical purity in a 23 f 2% radiochemical yield (n=14) (EOS) and estimated molar activity of 5.5±0.4 GBq/μmol.

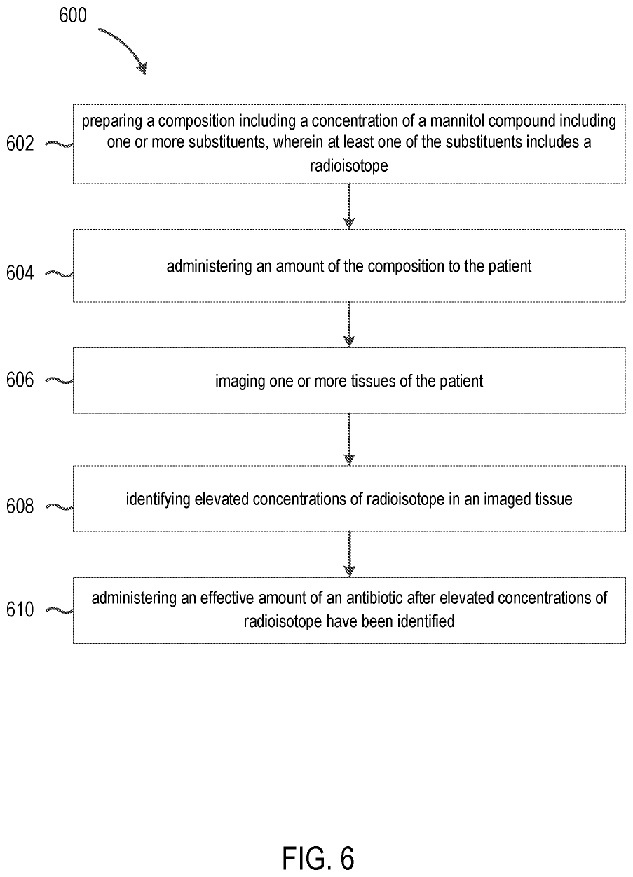

Referring now to FIG. 6, in some embodiments, the present disclosure is directed to a method 600 of treating a pathogenic bacterial infection in an individual, e.g., a patient. At 602, a composition is prepared that includes a concentration of a mannitol compound. As discussed above, in some embodiments, the mannitol compound includes one or more substituents. In some embodiments, the mannitol compound includes a plurality of substituents. In some embodiments, at least one of the substituents includes a radioisotope. In some embodiments, at least one of the substituents includes a plurality of radioisotopes. In some embodiments, the radioisotope includes a halogen isotope. In some embodiments, the radioisotope includes a fluorine isotope. In some embodiments, the radioisotope includes [¹⁸F]. In some embodiments, the mannitol compound includes a chemical structure according to Formula III above.

At 604, an amount of the composition is administrated to the patient. In some embodiments, the composition is administered via any suitable method, e.g., orally, parenterally, etc., or combinations thereof. As discussed above, in some embodiments, when the radiopharmaceutical is administered to an individual having or suspected of having a bacterial infection, the radiopharmaceutical in the composition is taken up by and accumulates in the bacteria. In some embodiments, the patient is positive for sickle cell disease. At 606, one or more tissues of the patient are then imaged to identify the presence and location of the radioisotopes, and thus a bacterial infection. In some embodiments, the radioisotopes are imaged via any suitable imaging process, e.g., PET. At 608, elevated concentrations of radioisotope in an imaged tissue are identified. As discussed above, in some embodiments, the pathogenic bacterial infection includes E. coli, S. aureus, A. baumannii, S. epidermis, P. mirabilis, S. enterica, K. pneumonia, E. faecium, E. cloacae, M. marinum, or combinations thereof. In some embodiments, at 610, an effective amount of an antibiotic is administered after elevated concentrations of radioisotope have been identified. In some embodiments, imaging 608 is performed at intervals over a period of time to monitor the progress of a course of treatment, e.g., administration 610, against the infection.

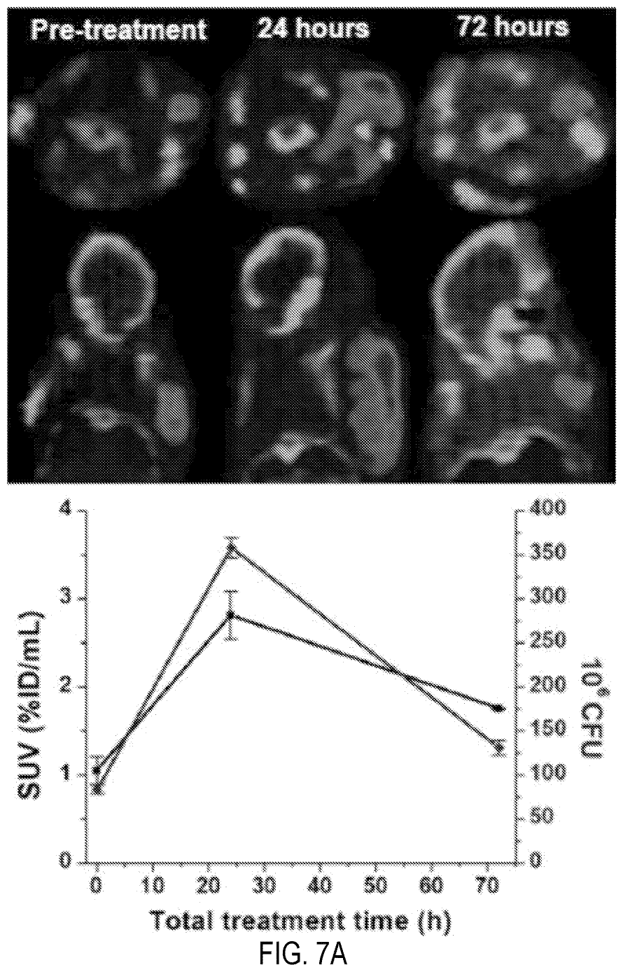

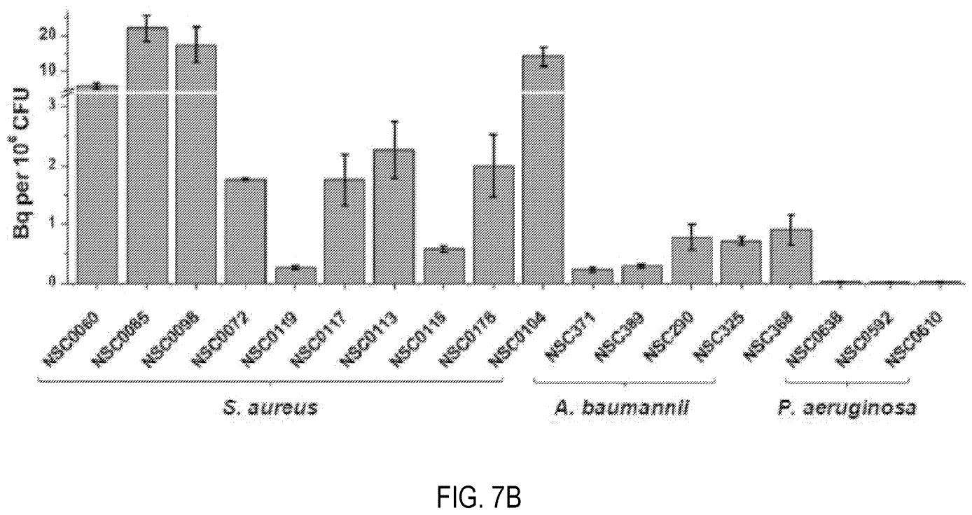

A growing incidence of antimicrobial resistance in many bacterial pathogens is a serious concern, as treatment failure is a major threat to global health. Inappropriate antibiotic use is also the primary driver of antibiotic resistance, which places undue risk onto patients for adverse events such as allergic reactions and C. difficile infection. Referring now to FIG. 7A-7B, exemplary embodiments of the present disclosure were prepared to demonstrate the ability of [¹⁸F]FMtl to quantify the efficacy of antibiotic therapy in vivo. Mice were inoculated with S. areus in the right triceps brachii and imaged 8 hours post-infection with [¹⁸F]FMtl, prior to initiation of vancomycin treatment (100 mg/kg every 8 hours, IP) and subsequently imaged at 24 and 72 hours post-treatment. PET signal diminished over the course of treatment, correlating closely with CFU burden (see FIG. 7A). The accumulation of [¹⁸F]FMtl was further investigated in a panel of bacterial isolates from infected combat wounds of military service members (see FIG. 7B).

Imaging can realistically play a complementary role in managing a number of clinical applications of infection with diverse etiologies. However, the complementary role imaging will play is ultimately limited to the in vivo sensitivity of the agent (CFU/mL). Several radiopharmaceuticals have been studied including glucose ([¹⁸F]FDG), sorbitol ([¹⁸F]FDS), and maltose (¹⁸F-fluoromaltose, ¹⁸F-fluoromaltotriose). [¹⁸F]FDS has shown adequate sensitivity to infection in both preclinical models and human disease; however, this agent is limited to the detection of Enterobacterales. Maltose-derived radiopharmaceuticals demonstrated improved strain coverage that includes P. aeruginosa and S. aureus; however, the sensitivity of these agents for clinically relevant concentrations of bacteria beyond E. coli remains uncertain. Other imaging agents, such as those targeting folate biosynthesis or transpeptidases, report limited (10⁸ CFU) or unknown sensitivity.

Deep-seated infections, like osteomyelitis, are very challenging to diagnose and also determine when treatment is working. Further, differentiating occlusion-associated events from bacterial infection in patients with sickle cell disease is an enormous and frequent challenge using the current standard of care.

[¹⁸F]FMtl was able to reliably detect 10⁵ CFU in vivo using the clinical standard SUV, which is of sufficient sensitivity for detecting an abscess. Furthermore, the sensitivity of [¹⁸F]FMtl did not diminish between E. coli and S. aureus, so sensitivity is not dependent upon a specific genus or family of bacteria. Compositions consistent with embodiments of the present disclosure useful as a tool to image a variety of clinically-relevant pathogens, as well as improve the management of deep-seated and difficult to manage bacterial infections commonly associated with these pathogens, such as osteomyelitis and prosthetic joint infection, even in patients with sickle cell disease.

Systems and methods of the present disclosure are advantageous in that they rapidly, specifically, and non-invasively detect a broad-spectrum class of bacterial infections in vivo. Embodiments of the present disclosure include positron-emitting analogues of mannitol, e.g., [¹⁸F]FMtl as a specific precursor for bacterial metabolism and a subsequent suitable imaging agent for in vivo use with PET. Some embodiments of the present disclosure include simple, widely-deployable radiosynthesis methods of radiolabeled mannitol compounds, e.g., [¹⁸F]FMtl. These compounds demonstrate “broad spectrum” bacterial specificity both in vitro and in vivo. In some embodiments, [¹⁸F]FMtl is produced using a simple nucleophilic substitution reaction that is deployable on virtually any commercially available synthesizer and is thus widely available for clinical use. The production of [¹⁸F]FMtl is straightforward, robust, and high-yielding and thus, facilitates wide accessibility.

It is demonstrated herein that [¹⁸F]FMtl can quantify antimicrobial treatment success in a murine myositis infection model. [¹⁸F]FMtl PET demonstrated high sensitivity and specificity for both gram-positive and gram-negative organisms in vivo and PET signal was shown to closely correlate with CFU burden. The [¹⁸F]FMtl signal is not limited by bacterial genus in vivo, correlates with CFU burden, and can quantify antimicrobial efficacy. Compositions consistent with embodiments of the present disclosure, e.g., including [¹⁸F]FMtl, can serve as a diagnostic tool for imaging infections in a diverse spectrum of pathogenic organisms, including S. aureus, A. baumannii, and E. coli. Accordingly, [¹⁸F]FMtl is able to be rapidly translated to clinical studies to serve as a highly sensitive non-invasive diagnostic tool to identify pathogens in vivo. Further, this tool facilitates rapid delineation of infection from sterile inflammatory processes, ultimately reducing the incidence of antimicrobial resistance promoted by selection pressures derived from unnecessary use of antibiotics.

Infectious disease remains the main cause of morbidity and mortality throughout the world. Of growing concern is the rising incidence of multidrug-resistant bacteria, derived from various selection pressures. Many of these bacterial infections are hospital-acquired and have prompted the Center for Disease Control and Prevention (CDC) in 2019 to reclassify several of these pathogens as urgent threats, its most perilous assignment. Consequently, there is an urgent need to improve the clinical management of bacterial infections by rapidly and specifically identifying bacteria and monitoring antibiotic efficacy in vivo. Clinical management of infection typically commences with empiric antibiotic therapy using broad-spectrum antibiotics, often combined with a targeted antimicrobial. The inability to rapidly delineate bacterial infection promotes unnecessary exposure to antibiotics, contributing to the rising incidence of antimicrobial resistance mechanisms and morbidities associated with antibiotic therapy. The inappropriate use of antibiotics is the primary driver for the development of antibiotic resistance mechanisms; this is alarming as the CDC estimates approximately 30% of prescribed antibiotics are unnecessary.

Treatment generally continues until biopsy/culture reveals the causative organism; however, treatment may continue in lieu of positive identification. Biopsy uses direct tissue sampling, which poses a risk of sample contamination, is prone to sampling error, is temporally limited to a single time point, and results are significantly delayed compared to a PET/CT scan. In addition, the cost of a biopsy, unnecessary antibiotics and extended hospital stays, in some cases, may be more expensive than the typical PET/CT scan.

As shown herein, [¹⁸F]FMtl detected and differentiated infection in as little as 5 minutes post-injection and using simple intravenous injection. This diagnostic test greatly reduces the potential complexity, cost, and delay for results associated with biopsy. Bacterial CFU were shown to correlate with PET SUV during vancomycin treatment using [¹⁸F]FMtl imaging (see FIG. 7A). In addition, [¹⁸F]FMtl demonstrated indistinguishable accumulation in E. coli and S. aureus in vivo. The compositions consistent with embodiments of the present disclosure are well-positioned to serve as valuable tools for diseases that are currently challenging to impossible to definitively delineate using current clinically-available imaging tools, such as degenerative disc disease (sterile inflammation) from discitis osteomyelitis (infection). With the embodiments of the present disclosure, imaging can rapidly diagnose infection ([¹⁸F]FMtl—broad spectrum) and optimize the selection of appropriate antibiotic for the pathogen ([¹⁸F]FDS—Enterobacterales specificity). Thus, these precision medicine tools may improve management of patient care and limit or eliminate unnecessary antibiotic use.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A composition for identifying a pathogenic bacterial infection, comprising:
a mannitol compound including one or more substituents, wherein at least one of the substituents includes a radioisotope.

2. The composition according to claim 1, wherein the mannitol compound includes a chemical structure according to Formula H: 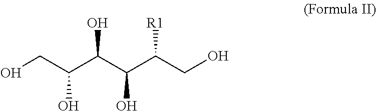
wherein R1 includes at least one radioisotope.

3. The composition according to claim 2, wherein the at least one radioisotope includes a halogen isotope.

4. The composition according to claim 3, wherein the at least one radioisotope includes [¹⁸F].

5. The composition according to claim 4, wherein the mannitol compound includes a chemical structure according to Formula III:

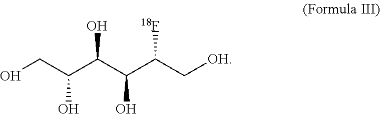 [SMILES: OC[C@@H](O)[C@@H](O)[C@H](O)[C@H]([18F])CO]

6. The composition according to claim 1, wherein the composition includes:
one or more additional active ingredients; or
one or more pharmaceutically acceptable adjuvants, diluents, excipients, carriers, or combinations thereof.

7. A method of treating a pathogenic bacterial infection in a patient, comprising:
preparing a composition including a concentration of a mannitol compound including one or more substituents, wherein at least one of the substituents includes a radioisotope;
administering an amount of the composition to the patient;
imaging one or more tissues of the patient; and
identifying elevated concentrations of radioisotope in an imaged tissue.

8. The method according to claim 7, wherein the radioisotope includes a halogen isotope.

9. The method according to claim 8, wherein the radioisotope includes [¹⁸F].

10. The method according to claim 9, wherein the mannitol compound includes a chemical structure according to Formula III:

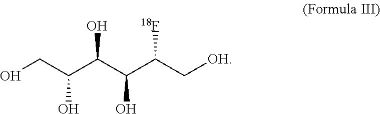 [SMILES: OC[C@@H](O)[C@@H](O)[C@H](O)[C@H]([18F])CO]

11. The method according to claim 7, further comprising:
administering an effective amount of an antibiotic after elevated concentrations of radioisotope have been identified.

12. The method according to claim 7, wherein imaging one or more tissues of the patient includes Positron Emission Tomography (PET).

13. The method according to claim 7, wherein the pathogenic bacterial infection includes E. coli, S. aureus, A. baumannii, S. epidermis, P. mirabilis, S. enterica, K. pneumonia, E. faecium, E. cloacae, M. marinum, or combinations thereof.

14. The method according to claim 7, wherein the patient is positive for sickle cell disease.

15. A method of making a composition for identifying a pathogenic bacterial infection, comprising:
providing a reaction medium including a concentration of a mannose compound including one or more substituents, wherein at least one of the substituents includes a radioisotope; and
converting the mannose compound to a mannitol compound via hydride-mediated reduction of the mannose compound.

16. The method according to claim 15, wherein the radioisotope includes a halogen isotope.

17. The method according to claim 16, wherein the radioisotope includes [¹⁸F].

18. The method according to claim 17, wherein the mannose compound includes 2-[¹⁸F]fluoro-2-deoxy-mannose ([¹⁸F]FDM).

19. The method according to claim 18, wherein providing a reaction medium including a concentration of a mannose compound including one or more substituents includes:
providing a precursor reaction medium including a concentration of a precursor compound including 4,6-O-Benzylidene-3-O-ethoxymethyl-2-O-trifluoromethanesulfonyl-1-O-methyl-β-D-glucopyranoside; and
forming the mannose compound by performing a substitution reaction at at least one carbon in the precursor compound to attach the radioisotope as a substituent of the at least one carbon.

20. The method according to claim 18, wherein the mannitol compound includes the chemical structure according to Formula III: 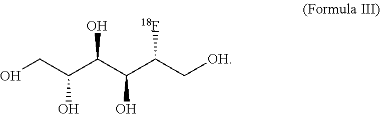 [SMILES: OC[C@@H](O)[C@@H](O)[C@H](O)[C@H]([18F])CO]
